(19) United States
(12) Patent Application Publication
(10) Pub. No.: US 2023/0253124 A1
(43) Pub. Date: Aug. 10, 2023

(54) Method for machine-assisted automated continuation of conversations between the user, software system, and health expert.

(71) Applicant: OptraHEALTH Inc., San Jose, CA (US)

(72) Inventors: Abhijeet Sharadchandra Gholap, Los Gatos, CA (US); Gauri Gholap, Los Gatos, CA (US); Ashwin Kotwaliwale, Saratoga, CA (US); Nitin Sharma, Saratoga, CA (US)

(73) Assignee: OptraHEALTH Inc., San Jose, CA (US)

(21) Appl. No.: 17/993,772

(22) Filed: Nov. 23, 2022

(51) Int. Cl. G16H 80/00; G16H 40/67

(52) U.S. Cl. CPC G16H 80/00; G16H 40/67

(57) ABSTRACT

Method for machine-assisted automated continuity of conversation between user and software system by identifying parts of the conversation that should be handled by a human health expert. A user utilizes a software system such as virtual assistants, chatbots, voicebots, etc., for working in a specific medical scenario such as counseling, data intake, education, Tele-Health, etc., and the method determines checkpoints when a human health expert should address the users' questions/queries. The method uses a medical scenario classifier to map the user's input. Medical scenarios are tagged if they should be handled by a health expert. A medical scenario database is built and continually updated by ingesting medical literature, ontologies, knowledgebases, etc. A health expert can restore the conversation between the user and the software system as required.

### FIELD OF INVENTION

In general, the present invention relates to the field of software systems used in TeleHealth, health IT, patient education, counseling, etc.

### BACKGROUND OF THE INVENTION

Software systems powered by computational linguistic algorithms are engaging users more meaningfully. Thus, enabling conversational information retrieval using interactive user interfaces (such as chatbots, voicebots, web and mobile apps, etc.). Such systems have gained popularity in healthcare where users are increasingly using such conversational tools. A few examples where such systems are used are for educating users about medical topics, for an explanation of medical documents, for users to provide information to their providers, and payors, during TeleHealth consults, and in other health IT applications. Generally, conversational systems are coupled to a backend database from which information is retrieved and presented to the user, powered by semantic mapping, keyword search, etc., to understand the user's input. However, several scenarios are not efficiently handled by conversational systems. Scenarios such as, when a user's input is complex or when a sensitive medical topic is encountered, such scenarios should be answered by health experts. A few examples of such questions are “Am I going to get cancer?” or “I am having side effects; can I take another medication?” etc. Additionally, users will echo the same question in a variety of different ways. In such cases, the conversational system is unable to process the user's request further. This leaves the user confused and additionally the user's query is not answered. Several present-day automated conversational systems functions in this manner and even fewer conversational systems are trained to understand medical information.

This method describes the process of understanding medically relevant user inputs and providing a complete resolution to a scenario in the healthcare domain by providing automatic continuity of the conversation by engaging the user with health experts, additionally providing health experts functionality to restore user and system conversation.

### DETAILED DESCRIPTIONS OF THE INVENTION

The field of computational linguistics has evolved significantly in the past decade. Computer software-based systems are leveraging this advancement, especially in healthcare. Such systems use artificial intelligence (AI) based conversationality, which is designed to provide information to users and collect information from users. Such systems use an AI core and natural language processing (NLP), deep learning, and language understanding/processing algorithms to accomplish their aims. Conversational AI systems designed specifically for the healthcare domain are lesser in number as compared to other domains.

A practical problem that is often encountered in conversational systems used for TeleHealth consults and in other health IT applications is bringing complete resolution to the user's questions. In the absence of a resolution, users abandon their session leading to undesired outcomes.

Our invention solves this problem by providing accurate and complete information to the user by automatically forwarding certain scenarios to human experts, encountered in TeleHealth and other health applications. This method enables the continuity of conversation between a user, conversational system, and health experts, for scenarios when the conversational system is unable to provide a resolution to the user's medical conversations. The embodiments of this invention implement methods to intelligently understand the user's input, determine if accurate responses are available, and in the absence of which, automatically transfer the conversation to health experts (human agents). Scenarios where this invention is valuable, are when the users ask complex questions, when the questions indicate medical severity and complexity or when answers are unavailable, or when a question should be specifically handled by a health expert.

As medical conversations contain a plethora of medical (technical) terms, the meaning of input must be carefully understood, as even a slight change in the sequence of words may change the meaning of the input, thus leading to an unresolved scenario.

In our method, a Natural Language Processing process starts by analyzing the users' inputs by identifying the sentiment and underlying semantics. Such inputs are then validated for accuracy and a severity/complexity threshold score is generated. If the threshold score is above an arbitrary value, then a database match is executed. The database used is a medical scenario database which will help determine further processing of the user's input.

This medical scenario database comprises clusters of medical scenarios that should be handled by a health expert. If the user's input is mapped to a cluster with a pre-defined level of confidence that indicates the need for health expert intervention, then the conversation is automatically transferred to the next available health expert. The user continues their conversation with the health expert, which when terminated is redirected back to the automated system. Additionally, the health expert may also restore the conversation between the user and the system.

Multiple methods are used to build the medical scenario database cluster by analyzing a plurality of medical information from a variety of sources. By applying techniques such as medical text analytics, ontologies, etc., we identify medical topics such as severity, complexity, certain diagnosis types, treatment options, immediate support, mental health issues, etc., that require health expert(human) intervention. This method uses the intellectual property (U.S. Ser. No. 10/754,882B2) granted to the authors of this patent, which enables converting any medical document into a knowledgegraph using a machine-assisted approach. This approach greatly provides the functionality to ingest and analyze any medical document for downstream processing such as creating this medical scenario database.

In summary, the invention enables automated conversation continuation between a user, system, and health experts. This leads to a complete resolution of users' medical questions increasing user/patient satisfaction.

### Applications

Our invention finds applicability in medical conversational systems. Such systems commonly use chatbots, voicebots, mobile and web apps, etc., for user interactions. Several user scenarios cannot be handled by such systems or have a medical complexity/severity associated with, that which should be handled by health experts. Our invention solves this problem by enabling continuity of conversation between a user, conversational system, and health experts.

### REFERENCES

The systems defined in the patent by Henry (US20190012390A1) refer to providing a live agent with more information about a conversation and the patent assigned to Conway (U.S. Ser. No. 10/194,029B2) is for analyzing data from online forums using computational linguistics. A patent granted to Nishant (July 2017) describes querying disconnected websites using conversational approaches and patent issues to Whitecotten in (U.S. Ser. No. 10/694,038B2) describe routing voice calls using context analysis in call centers. A patent was issued to Gholap et at (U.S. Ser. No. 10/754,882B2), also the authors of this patent describe a method that converts any medical document into a knowledgegraph. Such is used to generate the medical scenario database cluster.

The following table lists other such references

| | | |
|---|---|---|
| U.S. Pat. No. 10,754,882B2 | Aug. 25, 2018 | Gholap et al. |
| U.S. Pat. No. 10,027,618B2 | Feb. 4, 2016 | Harasimiuk et al. |
| US2016179323A1 | Jun. 23, 2016 | Kashi et al. |
| US2021081750A1 | Oct. 6, 2016 | Loftus et al. |
| U.S. Pat. No. 9,866,693B2 | Nov. 9, 2017 | Tamblyn. |
| US20170339274 | Nov. 23, 2017 | Odinak et al. |
| US2018001223 | Jan. 11, 2018 | Sapoznik et al. |
| US20180013699 | Jan. 11, 2018 | Sapoznik et al. |
| US20180048865 | Feb. 15, 2018 | Taylor. |
| US20180054523 | Feb. 22, 2018 | Zhang et al. |

In summary, as of this writing, no prior art has been identified that performs continuation of medical conversations between a user, conversational system, and health experts.

### BRIEF DESCRIPTION OF THE DRAWINGS

FIG. 1 is a block diagram that illustrates the steps of the method that provides the automated continuation of the conversation between a user and health experts.

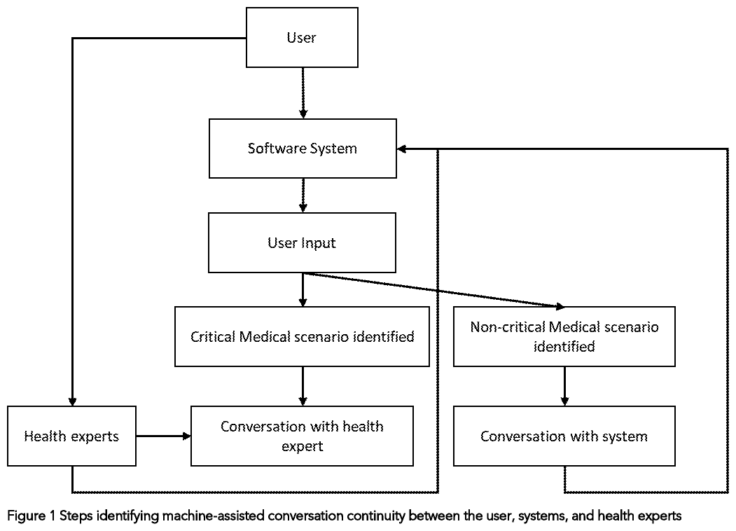

FIG. 2 is a block diagram that illustrates all the processors required for determining conversation meta-data to further determine criticality.

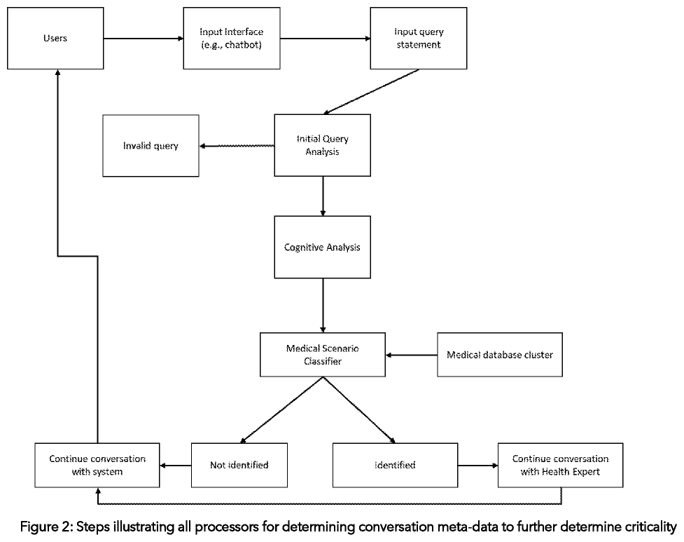

FIG. 3: is a block diagram that displays the process for generating medical database clusters used for storing specific medical scenarios that require conversation redirection and continuity with health experts.

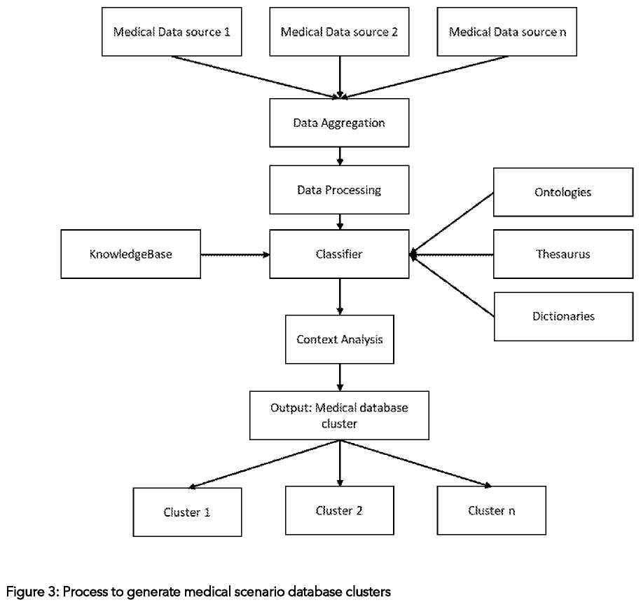

What is claimed:

1. Method for machine-assisted automated continuation of conversations between a user, software system, and health expert:
A: A system comprising a user interface coupled to a cognitive engine and a medical scenario database cluster.
B: A method to validate if the user's inputs are medically relevant.
C: A method to perform language analysis of the user's input using:
Sentiment analysis
Contextual identification of medical terminologies such as severity etc., specific medical scenarios, input question complexity, and other such topics.
D: Annotating the user's input for:
Context
Semantics
Ontologies
Medical terms
Medical phrases
E: Mapping the analyzed inputs to the medical database cluster.
F: Establishing a scenario assessment score based on combining information from
Mapping relevance
Medical database cluster
A plurality of ontologies, thesaurus, etc.
G: Based on the relevance, either automatically routing the conversation to available health experts or automatically providing a response to the user and allowing the user to communicate with the conversational system.
H: Providing optionality to the user to continue the conversation with the conversational system and/or health experts.
I: Building a medical database cluster to store medical scenarios that require redirection from a health expert. The said medical database clusters are built using a plurality of medical information sources such as
Signs & Symptoms
Medical disease severity
Drugs (e.g., life-threatening drugs)
Other health parameters
J: Sending the context of the conversation between the conversational system and users to health experts, thus enabling more meaningful conversations between the user and health experts.
K: Storing anonymized conversations that occurred between the users and health experts for model training.

2. Method for machine-assisted automated continuation of conversations between a user, software system, and health expert as claimed in claim 1 further comprises the steps:
A text-voice conversational system that is managed by a command
Allows the user to enter queries in natural language
Validate the user's queries for medical accuracy, providing initial acceptance or rejection.

3. Method for machine-assisted automated continuation of conversations between a user, software system, and health expert as claimed in claim 1 further comprises the steps:
A pre-processor that identifies medical keywords
A processor that extracts semantics, hidden relationships in the user input
A processor that annotates user queries based on pre-trained data models for identifying and assigning
Severity score
Contextuality
Intent
Hidden medical terms and phrases

4. Method for machine-assisted automated continuation of conversations between a user, software system, and health expert as claimed in claim 1 further comprises the steps of
A processor for mapping the user's input meta-data to pre-defined database cluster meta-data.

5. Method for machine-assisted automated continuation of conversations between a user, software system, and health expert as claimed in claim 1 further comprises the steps of
A processor that determines the scenario assessment score
A processor that outputs the scenario assessment score
To continue the automated conversation
To redirect the conversation with a health expert

6. Method for machine-assisted automated continuation of conversations between a user, software system, and health expert as claimed in claim 1 further comprises the steps of
A processor that determines the scenario assessment score
A processor that outputs the scenario assessment score for:
To continue the automated conversation
To redirect the conversation to a health expert
To hand off the conversation back to the system and user.

7. Method for machine-assisted automated continuation of conversations between a user, software system, and health expert as claimed in claim 1 further comprises the steps of
A graphical user interface for a health expert to interface with user queries as received from a processor

8. Method for machine-assisted automated continuation of conversations between a user, software system, and health expert as claimed in claim 1 further comprises the steps of
A processor that generates medical scenario database clusters
By analyzing a plurality of health records, literature, and such medical information.

9. Method for machine-assisted automated continuation of conversations between a user, software system, and health expert as claimed in claim 1 further comprises the steps of
A processor to send the context of the users' conversation to a health agent before the start of user-health expert interaction

10. Method for machine-assisted automated continuation of conversations between a user, software system, and health expert as claimed in claim 1 further comprises the steps of
A processor for continually storing user-health expert interactions.
A processor for continual learning from user-health expert interactions
A processor for continual re-annotating scenario database clusters
